(19) United States
(12) Patent Application Publication
(10) Pub. No.: US 2006/0234243 A1
(43) Pub. Date: Oct. 19, 2006

(54) Sample preparing unit

(76) Inventor: Lukas Bestmann, WINTERTHUR (CH)

Correspondence Address: SHOEMAKER AND MATTARE, LTD, 10 POST OFFICE ROAD - SUITE 110, SILVER SPRING, MD, 20910, US

(21) Appl. No.: 10/563,737

(22) PCT Filed: Jul. 3, 2004
(86) PCT No.: PCT/EP04/07284

§ 371 Date: Feb. 16, 2006

(30) Foreign Application Priority Data
Jul. 15, 2003 (EP) 03016057.6

(51) Int. Cl. C12Q 1/68; C12M 1/34

(52) U.S. Cl. 435/6; 435/287.2

(57) ABSTRACT

The invention relates to a unit for preparing reaction mixtures for chemical reactions, in particular polymerase chain reactions. The inventive unit comprises an input, output and at least one membrane. Said invention is characterised in that a compound bound to the membrane contains substances necessary for carrying out chemical reactions. A method for preparing reaction mixtures for chemical reactions, in particular polymerase chain reactions and a sample preparing device provided with said unit are also disclosed.

The present invention relates to a unit for preparing reaction mixtures, in particular polynucleotides for a chemical reaction such as the PCR reaction (polymerase chain reaction), and to a method for preparing such reaction mixtures using such a unit.

The isolation and analysis of polynucleotides such as DNA and RNA plays a central part in microbiological and genetic diagnosis. Isolation and/or purification of polynucleotides is necessary in order to obtain reliable results in the identification of genes in cells, of bacteria or viruses in a sample or the detection of mutations.

Conventional methods for preparing polynucleotide-containing samples comprise the step of lysis of cells to be investigated, and subsequent separation of interfering cellular constituents from the polynucleotides. The known separation methods can be arranged into two general categories. In one category of separation methods a polynucleotide-containing solution is purified by removing intefering constituents by, for example, precipitation and/or centrifugation of the polynucleotides (liquid phase purification). In the other category of separation methods the polynucleotides are bound to a solid support, and the interfering constituents in the sample are removed by washing with suitable solvents (solid phase purification).

The commonest methods for liquid phase purification of polynucleotide samples comprise the following three steps:
1. Lysis of cells to release the polynucleotides
2. Removal of interfering constituents in the sample from the liquid mixture
3. Concentration of the remaining mixture (e.g. by alcoholic precipitation) and redissolving of the polynucleotides

The second step can be effected, for example, by centrifugation, extraction with organic solvents, precipitation or chromatography.

The commonest methods for solid phase purification of polynucleotide samples comprise the following 4 steps:
1. Lysis of cells to release the polynucleotides
2. Binding of the polynucleotides on a solid support
3. Removal of interfering constituents in the sample by washing with suitable solvents
4. Elution of the polynucleotides from the solid support

Examples of proposed solid supports are membrane filters, metal oxides, latex particles or magnetic beads.

The methods described above have some disadvantages. It is usually necessary to carry out a plurality of steps of the methods in different apparatuses. In addition, these methods require the use of various solvents, some of which are not innocuous, such as phenols or halogenated solvents. Conventional methods are time-consuming, complicated and require for the purification the use of substances which must subsequently be removed again as quantitatively as possible because they impede subsequent chemical reactions such as the polymerase chain reaction (PCR) and subsequent analysis of the reaction mixtures.

Attempts to simplify the method for preparing polynucleotide-containing reaction mixtures, especially for PCR, have been described. WO 99/39009 describes a method in which the sample to be purified is initially brought into contact with a solid support in a unit. A lysing agent is bound to this solid support, for example a membrane or a filter. The lysis thus takes place on this solid support. Subsequently, the DNA present in the mixture is purified by treating the mixture with a DNA purifying agent such as an organic solvent. In the case where the DNA is bound to a solid support in this method, the polynucleotide is subsequently eluted from the support. Purification of the reaction mixture with organic reagents is necessary in this method. In addition, a composition comprising a chelating agent is used as eluent. Such substances interfere with the PCR and subsequent detection to a considerable degree, or completely prevent such subsequent steps.

WO 95/02049 describes a method in which a sample to be analyzed is initially filtered in a unit in order to remove liquid constituents. Subsequently, cells collected on the filter are resuspended and lysed. The lysed mixture is subsequently filtered into a second cartridge chamber. The filtered lysate is put onto a solid matrix in this chamber under conditions under which the DNA binds to the solid matrix. The solid matrix is, according to the examples of WO 95/02049, an ion exchanger resin. This resin is provided in the second chamber of the cartridge in the form of a suspension. The suspension is treated with washing solutions in order that interfering constituents are separated out and removed by a filter. Subsequently, the DNA is removed from the matrix with an eluent and likewise washed out of the cartridge through the filter. It is thus necessary in this method likewise to use a plurality of different solutions for suspending and washing the reaction mixture.

It is intrinsic to all the methods described above that they provide only one purified polynucleotide. There has been no description of a method or a cartridge by which a complete reaction mixture can be provided for PCR in a single operation.

The object of the present invention was to provide a unit and a method in order to provide a polynucleotide-containing reaction mixture for PCR in a simple manner from a biological sample.

The above object is achieved according to the present invention by a composition for carrying out a chemical reaction, a unit and a method, and an apparatus as defined in the independent claims.

The present invention relates to a unit, preferably a cartridge, for preparing reaction mixtures for chemical reactions, in particular for the polymerase chain reaction, comprising an inlet and an outlet and at least one support, preferably a membrane, characterized in that a composition, which is defined in detail hereinafter and which comprises substances necessary for carrying out the chemical reaction, is bound in solid form to at least one support.

The unit of the invention has the advantage that a reaction mixture for a chemical reaction can be provided in a simple and rapid procedure. The substance to be analyzed is put into the inlet of the unit. When the substance passes through the support which is present in the unit and which is preferably a membrane, it is mixed with a composition which is defined in detail hereinafter and which is preferably provided on the underside of a membrane. Thus, the finished reaction mixture emerges from the outlet of the unit. The composition is according to the invention preferably a lyophilizate.

The present invention is not restricted to a particular type of sample preparation unit. In principle, the present invention encompasses all units in which a sample can come into contact with a support to which a composition is bound. Units which are preferred according to the invention allow the sample to flow through in the vertical direction. It is particularly preferred according to the invention for the unit to be cartridge.

A support means according to the present invention any solid material to which the composition can be bound. For example, the support may be a membrane, one or more beads or a loose layer. The support is preferably according to the invention a membrane. A membrane is intended to mean according to the present invention a wall or thin layer which is at least partly permeable for liquids and solids. The membrane is particularly preferably a filter.

The speed at which the sample passes through the unit can be increased by applying an elevated pressure or reduced pressure to the unit. This can be achieved by providing appropriate apertures in the unit. If, for example, the sample is to be forced faster through the membrane with the aid of elevated pressure, an aperture for applying an elevated pressure should be provided in the region of the unit from the inlet to the membrane. The aperture may also be the inlet of the unit itself. An appliance for generating an elevated pressure can be connected to the unit at this aperture. This appliance may be for example a conventional pump. However, it is also possible to attach a syringe with whose aid it is possible to force air into the unit. If the sample is to be drawn faster through the membrane with the aid of reduced pressure, an aperture for applying a reduced pressure should be provided in the region of the unit from the membrane to the outlet of the unit. An appliance for generating a reduced pressure can be connected to the unit at this aperture. This appliance may be for example a conventional vacuum pump. However, it is also possible to attach a syringe with whose aid air can be drawn out of the unit. The pressure elevation or pressure reduction to be applied can easily be determined by the skilled worker.

A sample means according to the present invention the composition to be processed. This comprises, in the case of the preparation of reaction mixtures for a PCR, polynucleotides. The sample may be body fluid, for example blood or saliva, plant juice or a similar biological source or a synthetic composition.

A reaction mixture for the polymerase chain reaction (PCR) comprises for example besides the polynucleotide to be amplified, such as DNA, the enzyme polymerase to catalyze the amplification reaction, deoxyribonucleotide triphosphates (dNTPs) as building blocks for the polynucleotides to be synthesized, oligonucleotide primers to initiate the reaction, and where appropriate further substances such as buffers. The conventional preparation of a reaction mixture for PCR takes a not inconsiderable time because of the careful measurement of the amounts of the numerous constituents and the mixing. This expenditure of time and effort does not apply with the unit of the invention and the method of the invention.

The unit of the invention can be stored at room temperature (25° C.) for a long period. It is intended for single use.

With certain biological samples such as blood samples the mixing with the other constituents of the reaction mixture for the PCR must be preceded by the polynucleotide being released from the cells by lysis and purified from the other interfering constituents of the biological sample. This can likewise be carried out in a simple manner in one working step with the aid of a further embodiment of the unit of the invention. For this purpose, according to the present invention, additionally 1 or more membranes or supports, preferably 4 membranes, are provided in the unit between the inlet and the support to which the composition is bound. These supports, preferably membranes, are preferably disposed so that their pore size becomes smaller as the distance from the inlet of the unit increases. It is possible in this way for there to be a stepwise removal of increasingly small constituents from the sample without the risk of a membrane becoming blocked. Further purification can be achieved by providing a solid absorbing substance such as, for example, Aerosil in a space between two membranes.

In a preferred embodiment of the present invention, one of the additional supports is configured so that polynucleotides bind thereto. This can take place for example through the provision of appropriate functional groups on a membrane. It is preferred according to the invention to provide on a membrane functional groups which carry and/or are able to form a positive charge. Amino groups such as the diethylaminoethyl group may be mentioned as example. Other sample constituents can then be removed from the polynucleotides. The polynucleotides are subsequently eluted from the support, preferably the membrane, with the aid of an eluent. The eluent can in principle be introduced into the unit through the inlet or any other aperture present in the unit above the preceding support.

However, it is preferred according to the invention for the unit to comprise a unit for supplying a liquid such as the eluent. This unit has a storage container in which the eluent is stored. The storage container is separated from the interior of the unit by a membrane. When the membrane is intact, the eluent cannot enter the unit. This membrane can be destroyed by applying a reduced pressure, and the eluent thus enters the unit.

The membranes present in the unit are preferably according to the invention filters, i.e. materials suitable for filtration. Any conventionally used filter can in principle be used for the unit of the invention. The filters may be for example fabricated from cellulose, cellulose derivatives such as cellulose acetate, for example zeolite, plastics such as polyamides, for example nylon, polysulfones or halogenated polymers, for example polytetrafluoroethylene or polyvinyl chloride. The filters used in the unit of the invention preferably have pore sizes in a range from inclusively 5 μm to inclusively 0.2 μm, preferably 0.3 to 0.9 μm. A limitation to be taken into account in a preferred embodiment of the present invention is that the pore size of the filters used should gradually become smaller as the distance from the inlet of the unit increases.

The support provided for binding polynucleotides is preferably a filter and must be configured such that it is able, via interactions with polynucleotides, to bind these substances. It is possible to utilize for this purpose for example ionic interactions or van de Waal's forces. Ionic interactions are preferably employed according to the invention. Polynucleotides are negatively charged molecules at physiological pH. The desired binding of the polynucleotides to a membrane can be achieved by providing functional groups which have and/or are able to form a positive charge.

Conventional cation exchanger resins can be used for this purpose as material for the filter or for impregnating a filter made of the aforementioned materials. This membrane preferably has according to the invention amino groups such as diethylaminoethyl (DEAE) groups.

In a preferred embodiment of the present invention, one or more of the membranes or supports described above can be impregnated with a substance which can increase the surface tension of liquids. This leads firstly to an additional filter effect and secondly to an improved flow behavior of the reaction mixture. It is possible to employ all substances which have the appropriate effect on the surface tension and are suitable for impregnating a membrane. Polymeric silicon compounds are preferably used for this purpose according to the invention. Polydimethylsiloxane such as Dimeticon® are particularly preferably employed according to the invention.

In the case where units are present for purifying the sample, the unit of the invention further comprises a waste container to receive the sample constituents which are not intended to get into the reaction mixture. It is possible with the aid of a control unit such as a three-way tap alternatively to guide substance via appropriately provided supply lines into the waste container or into a collecting device for the reaction mixture.

If a lysis is necessary, this can alternatively take place by adding a lysing agent through the inlet or any other aperture present in the unit to the sample or by contacting the sample with a membrane impregnated with lysing agent.

It is possible with all embodiments of the present invention to provide a capillary above the inlet. This makes it possible to introduce a defined volume of a liquid sample into the unit.

The unit of the invention can be fabricated from any conventional material used for such units. The use of glass as material for the walls of the unit is preferred according to the invention.

The unit of the invention typically has size of about 3 cm.

The present invention further provides a method for preparing reaction mixtures for chemical reactions, in particular for the polymerase chain reaction, comprising the steps:
a) introducing a sample into the unit as claimed in any of claims 1 to 14;
b) passing the sample through a support, preferably a membrane to which is bound a composition which is defined in detail hereinafter and comprises the substances necessary to carry out the chemical reaction, so that the finished reaction mixture emerges from the outlet of the unit.

It is possible in this way to provide a reaction mixture within a short period of, typically, 3 to 5 minutes. The preparation of the reaction mixture with numerous measuring, pipetting, diluting and mixing steps, which is time-consuming and labor-intensive especially for PCR, is unnecessary in the method of the invention. The reaction mixture with the exception of the polynucleotides to be analyzed is provided in the form of a composition in solid form on a support, preferably a membrane. The composition is preferably a lyophilizate. In the case of a reaction mixture for PCR, this composition comprises the enzyme DNA polymerase to catalyze the amplification reaction, deoxyribonucleotide triphosphates (dNTPs) as building blocks for the polynucleotides to be synthesized, oligonucleotide primers for initiating the reaction and, where appropriate, further substances such as buffers. It is possible according to the invention to use for this purpose in principle all reaction mixtures which can be lyophilized. The compositions described in U.S. Pat. No. 5,861,251 and U.S. Pat. No. 6,153,412 may be mentioned as example.

A composition which can preferably be employed according to the invention for a PCR is the mixture described hereinafter. This comprises a polymerase, for example DNA polymerase, preferably Taq polymerase. Taq polymerase is a DNA polymerase derived from the organism Thermos aquaticus and is the fastest amplifying polymerase.

The nature of the polymerase used depends on the substance to be amplified. Appropriate polymerases for all respective targets are known to the skilled worker.

The polymerase is provided in a stock solution which additionally comprises a buffer solution, preferably Tris buffer having a pH of 8.3 and bovine serum albumin (BSA).

The stock solution is subsequently mixed with a buffer solution, at least one alkali metal and/or alkaline earth metal halide, the appropriate deoxyribonucleotide triphosphates (dNTPs), at least one primer, one stabilizer and substances for detecting the reaction product and, where appropriate, further additives. The solution is subsequently diluted where appropriate with water.

The buffer solution is preferably Tris buffer having a pH of 8.3. The buffer solution preferably contains in addition bovine serum albumin (BSA) and MgCl₂. It is moreover possible for additional substances such as, for example, Ficoll to be added to the buffer solution.

The composition comprises MgCl₂ as at least one metal halide. The composition may additionally comprise further alkali metal and/or alkaline earth metal halides, preferably chlorides, particularly preferably KCl.

The composition for the PCR must comprise at least one primer. A primer is an oligonucleotide sequence which adheres to the polynucleotide to be amplified and is necessary for the operation of the polymerase. Two primers are required in the case of amplification of double-stranded DNA. The primers are to be selected depending on the polynucleotide to be amplified. Appropriate primers are known to the skilled worker. Primers which consist of segments of 16SRNA are preferred according to the invention.

The preferred composition further comprises a stabilizer. The stabilizer stabilizes the polymerase during the lyophilization and enables the composition to be stored for a long time with negligible loss of activity of the enzyme. The lyophilization of proteins, and stabilizers used for this purpose are known. The stabilizer is responsible for maintaining the tertiary structure of the protein during the lyophilization. It is known that the tertiary structure varies for each protein. Reliable prediction of which stabilizer is suitable for which protein is thus impossible. It has been found that disaccharides and, particularly preferably, trehalose is suitable for stabilizing Taq polymerase. A composition with Taq polymerase as enzyme therefore preferably comprises according to the invention a disaccharide and preferably trehalose.

The preferred composition further comprises substances for detecting the reaction product. These substances are ones which can be detected by physical methods and bind or adhere to the amplified polynucleotide. It is thus possible by detecting the substance to establish the presence of the polynucleotide. Dyes are conventionally employed as such substances. Examples which may be mentioned are dyes for gel electrophoresis or dyes for fluorescence measurement. Substances which can be detected by fluorometry are preferred according to the invention. Such dyes are known to the skilled worker. Fluorescein, fluorescein isothiocyanates, LC Red 640, LC Red 705, Rhodamine R6G, Oregon 488, Rhodol Green or FAM are mentioned here as example.

However, it is also possible according to the invention to use components which are employed in the FRET (fluorescence resonance energy transfer pair) technology. This entails two different oligonucleotides which are connected to fluorophores and are referred to as sensor and anchor being added to the PCR reaction mixture. These oligonucleotides must be selected so that they are able to adhere to the amplified polynucleotide spatially close to one another. The anchor oligonucleotide is connected to a dye referred to as donor fluorophore. When the amplified reaction mixture is irradiated with electromagnetic radiation of appropriate wavelength, the donor fluorophore absorbs the incident electromagnetic radiation and transfers it to the spatially close acceptor fluorophore. The acceptor fluorophore is preferably bound to the sensor oligonucleotide and generates, because of the electromagnetic radiation transferred from the donor fluorophore, a detectable fluorescence emission.

Embodiments of FRET technology are described for example in U.S. Pat. No. 6,174,670, WO 92/14845 or EP-B-0 870 063.

A further possibility according to the present invention is to add Carbopol 940 to the composition in order to prevent oxidative effects.

The polymerase stock solution comprising the polymerase, the buffer solution and bovine serum albumin is mixed with a buffer solution with at least one alkali metal and/or alkaline earth metal halide, with at least one primer, and with a dye (in the case of use of FRET technology with an anchor oligonucleotide and a sensor oligonucleotide) and gentle stirring. Subsequently, the appropriate deoxyribonucleotide triphosphates (dNTPs), the stabilizer and, where appropriate, additional components are added. The mixture obtained in this way is subsequently lyophilized by standard methods known to the skilled worker.

The lyophilizate obtained in this way is notable for high storage stability. Virtually no loss of activity of the polymerase occurs.

Although the provision of the composition in the form of a lyophilizate represents the preferred embodiment of the present invention, it is also possible however to choose other possibilities known to the skilled worker in order to bind the above composition to the support.

A reaction mixture which is particularly preferred according to the invention comprises inter alia the following components in the following amounts:

| Component | Amount |
|---|---|
| Buffer solution of 50 mM Tris (pH = 8.3), | 2.0 μl |
| 0.25 mg/ml bovine serum albumin, 0.5-1.0% Ficoll | |
| and 1 mM MgCl₂ | |
| 50 mM KCl | 5.0 μl |
| 200 μM dNTP | 2.0 μl |
| Polymerase stock solution (0.4 U/10 μl | 2.0 μl |
| polymerase, 250 μg/ml bovine serum albumin) | |
| 0.5 μM primer 1 | 1.0 μl |
| 0.5 μM primer 2 | 1.0 μl |
| 0.2 μM sensor oligonucleotide | 0.2 μl |
| 0.4 μM anchor oligonucleotide | 0.4 μl |
| 1.0 mM MgCl₂ | 0.8 μl |
| Trehalose | 8% (w/v) |

While the sample passes through preferably the membrane, the composition which is particularly preferably located on the underside of the membrane is carried along with the sample liquid or dissolved therein. In this way, a finished reaction mixture emerges at the outlet from the unit and comprises, besides the polynucleotide to be amplified and to be investigated, the constituents of the composition.

The speed at which the sample passes through the membrane can be increased by applying an elevated pressure on the side of the membrane facing away from the outlet or applying a reduced pressure on the side of the membrane facing the outlet. This can be carried out by the measures described above.

The simple method described above can be carried out only with samples with which neither lysis of cells to release polynucleotides nor purification of the sample is required. However, with the clear majority of biological samples the aforementioned steps are necessary. In a preferred embodiment of the present invention, therefore, the method of the invention comprises as additional steps between steps a) and b)
a1) lysis of cells in the sample
a2) separation of the polynucleotides from other sample constituents.

The lysis of cells can be carried out by conventional methods of the prior art. In one embodiment of the present invention, a lysing agent is added to the sample present in the unit for this purpose. The first membrane in the unit subsequent to the inlet has a pore size such that cells cannot pass through. On the other hand, particles obtainable by lysis of the cells can get through this membrane.

A further embodiment of the present invention consists of the lysis step taking place in such a way that the first membrane in the unit subsequent to the inlet is impregnated with a lysing agent. When the cell-containing sample comes into contact with the membrane, the lysis of the cells is initiated.

Conventional lysing agents which can be used for the present invention include for example a detergent. It is possible to use anionic detergents such as N-lauroylsarcosine or sodium dodecyl sulfate. Enzymatic lysing agents can also be used. Proteinase K is mentioned as example thereof which is preferred according to the invention. However, lysing agents with constituents which interfere with the subsequent chemical reaction and detection are unsuitable for the method of the invention. For example, chelating agents interfere with a subsequent PCR and evaluation thereof. Lysing agents with chelating constituents should therefore be avoided in the preparation of reaction mixtures for PCR. The amount of lysing agent depends on the amount of cells present in the sample volume. An amount of 1-10 μl of lysing agent is preferably provided according to the invention.

In the method of the invention, the sample passes through a plurality of additional membranes or supports in the unit, depending on the characteristics of the sample and the purity requirements for the reaction mixture to be prepared. As described above, for this purpose additionally one or more membranes or supports, preferably 4 membranes, are provided in the unit between the inlet and the support to which the composition is bound. These membranes or supports are preferably disposed so that their pore size becomes smaller as the distance from the inlet of the unit increases. Stepwise removal of increasingly small constituents from the sample is possible in this way, without the risk of a membrane becoming blocked. The pore size and characteristics of the membranes are described above.

The purity of the polynucleotides present in the sample increases owing to the passage through the various membranes or supports. The disposition according to the invention of membranes or supports means that no washing steps are required in the method of the invention, in contrast to conventional methods from the prior art. Addition of washing solutions entails, besides the additional expenditure of effort, always the risk of contamination of the resulting reaction mixture. Such steps are therefore avoided according to the invention.

Further purification is possible if a solid-absorbing substance such as, for example, Aerosil is provided in a space between two membranes. It is possible in this way for constituents such as hemoglobin to be removed from a blood sample without a filter becoming blocked. This solid-absorbing substance is preferably disposed according to the invention between the first two membranes subsequent to the inlet of the unit.

In a preferred embodiment of the present invention, one or more of the membranes or supports described above can, as described above, be impregnated with a substance which can increase the surface tension of liquids. This leads firstly to an additional filter effect and secondly to an improved flow behavior of the reaction mixture. It is possible to employ all substances which have the appropriate effect on the surface tension and are suitable for impregnating a membrane. Polymeric silicon compounds are preferably used for this purpose according to the invention. Polydimethylsiloxanes such as Dimeticon® are particularly preferably employed according to the invention. Dimeticon® is a substance commercially available as medicament for gastric complaints. It has the chemical formula

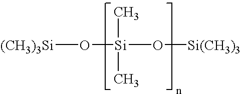

In a particularly preferred embodiment of the present invention, the step of separating the polynucleotides from other sample constituents includes the contacting of the sample with a support, preferably a membrane, which is provided for binding polynucleotides. The characteristics of this membrane have already been explained above. The polynucleotides which are negatively charged under the preparation conditions of the invention, owing to the provision on the membrane of functional groups which have and/or are able to form a positive charge, are according to the invention preferably bound to the membrane. It is possible to use for this purpose conventional cation exchanger resins as material for the filter or for impregnating a filter made of one of the abovementioned materials. It is preferred according to the invention for this membrane to have amino groups such as diethylaminoethyl (DEAE) groups.

The other sample constituents which do not bind to this membrane pass through the membrane and are thus separated from the polynucleotides bound to the membrane. In a preferred embodiment of the present invention, the sample constituents not bound to this membrane are passed to a waste container. A control unit such as a three-way tap is provided below the membrane which binds polynucleotides. When the sample is introduced into the unit, this tap is set so that it guides substance which passes through into the waste container.

After the sample has passed through the membrane which binds polynucleotides and guidance thereof into the waste container, the polynucleotides are, in this preferred embodiment of the present invention, eluted from the membrane. An eluent is put on the membrane for this purpose. In principle, the eluent can be introduced via the inlet or any other aperture present above the support into the unit. As stated above, however, it is preferred according to the invention for the eluent to be stored in a storage container of a unit for supplying a liquid and to be introduced into the unit by destroying a membrane by applying reduced pressure.

Conventional eluents can be employed according to the invention. The choice of the eluent depends on the characteristics of the membrane which binds polynucleotides and can be carried out routinely by the skilled worker. However, eluents with constituents which interfere with the subsequent chemical reaction and detection are unsuitable for the method of the invention. For example, chelating agents interfering with a subsequent PCR and evaluation thereof. Eluents with chelating constituents should therefore be avoided in the preparation of reaction mixtures for PCR. It is preferred according to the invention to employ a high salt solution as eluent. A high salt solution means according to the present invention an approximately 0.7-2M salt solution. However, 0.9-1.8M salt solutions are preferred as eluent. The eluent of the invention preferably has a pH of from 8.0 to 8.5, which can be achieved by adjustment with, for example, an alkali metal hydroxide such as sodium hydroxide solution. The amount of eluent depends on the amount of polynucleotides present in the sample volume. It is preferred according to the invention to provide an amount of 0.1-1.5 μl of eluent in the storage container.

In the method of the invention of this embodiment, after the other sample constituents have passed through the membrane which binds polynucleotides, the control unit such as the three-way tap is changed so that liquid now arriving is guided not into the waste container but through the support to which the composition is bound into an appropriate collecting device. In a preferred embodiment of the unit with storage container for the eluent, a reduced pressure is applied and thus the membrane of the unit for supplying the eluent is destroyed. Alternatively, the eluent is put into the unit through the inlet of the unit. The eluent reaches the membrane which binds polynucleotides and elutes the polynucleotides from this membrane.

The eluate containing the polynucleotides passes through the support, preferably the membrane, to which the composition is bound. The composition is taken up by the eluate and thus the finished reaction mixture is formed.

The finished reaction mixture emerges from the unit of the invention through the outlet and is collected in a suitable collecting device. Care must be taken to ensure in this case that the aperture for applying a reduced pressure, if such has been applied, is closed in good time in order to avoid the reaction mixture passing through this aperture.

The whole preparation procedure of the method of the invention is thus very fast and expends little effort. Typically, the finished reaction mixture is provided after 3-5 minutes. Since the entire process is carried out in one device, the risk of contamination associated with the method of invention is only low.

The unit of the invention and the method of the invention can also be used to process small sample volumes. The finished reaction mixture can typically have a volume of about 1 nl to 2 μl. It is, of course, also possible to process larger sample volumes, however, where appropriate with an adaptation of the abovementioned amounts of necessary eluent and lysing agent.

The unit of the invention is also suitable, because of the simple operation, for automated preparation of reaction mixtures.

The present invention further relates to a device for preparing reaction mixtures for chemical reactions, in particular for the polymerase chain reaction, comprising
a) at least one unit described above;
b) at least one reaction device which is connected via an aperture to the outlet of a unit and can be separated from the sample preparation device after charging with a reaction mixture.

In a preferred embodiment, a plurality of, for example 3, units such as, for example, cartridges are provided in a housing. The housing may be for example an article made of plastic such as a polycarbonate block. Each unit is connected via its outlet to a separate collecting device. It is possible in this way for example to prepare simultaneously a reaction mixture, a positive sample and a negative sample.

The solutions emerging from the units enter one or more reaction devices. Reaction devices are according to the present invention devices suitable for carrying out a chemical reaction such as PCR and advantageously also for carrying out a subsequent evaluation. Such reaction devices are described for example for PCR. Reference is made in this connection to U.S. Pat. No. 5,589,136, U.S. Pat. No. 5,639,423 and U.S. Pat. No. 5,958,349, whose corresponding contents are hereby expressly included in the present application. A further suitable reaction device is described in WO 03/019158, whose contents concerning this are hereby expressly included by reference.

The device is configured according to the present invention in such a way that the unit can be separated from the reaction device. One configuration permits the reaction device to be taken out of the sample preparation device. The reaction device is charged via the unit and then taken out and transferred into an apparatus for carrying out and, where appropriate, evaluating a chemical reaction such as a PCR. An alternative configuration permits the unit to be separated from the remaining device. In this case, the reaction device may also be present in an apparatus for carrying out and, where appropriate, evaluating a chemical reaction such as a PCR, while it is charged via the unit. The unit is then separated from the reaction device.

The present invention is illustrated below by means of non-restrictive drawings and examples. These show:

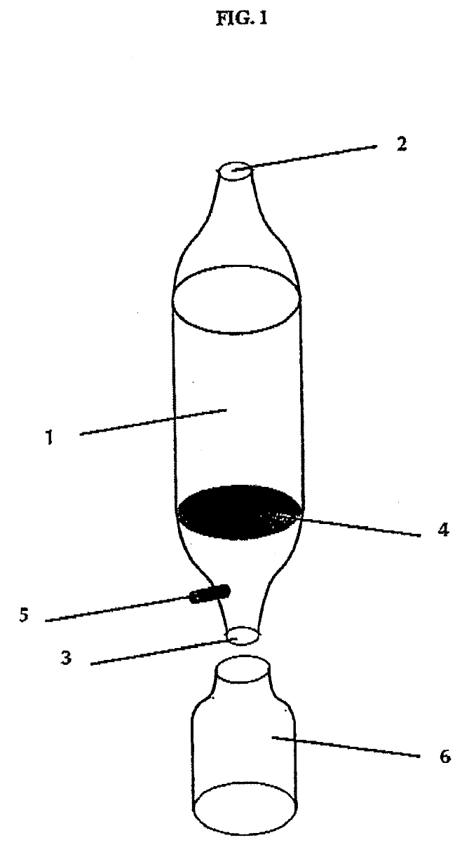

FIG. 1 a first embodiment of the unit of the invention

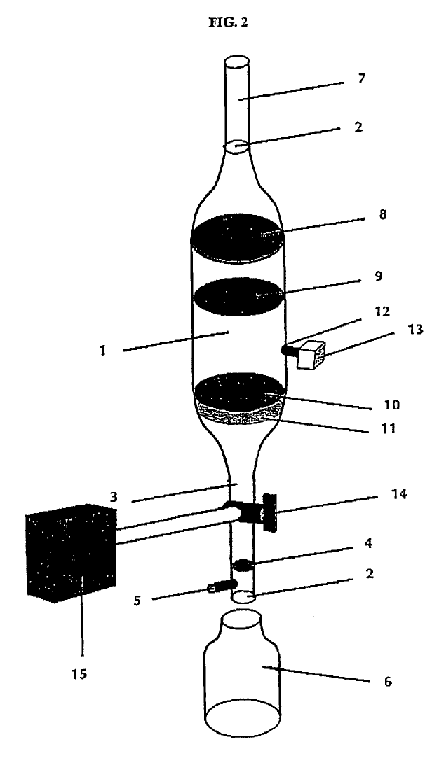

FIG. 2 a second embodiment of the unit of the invention

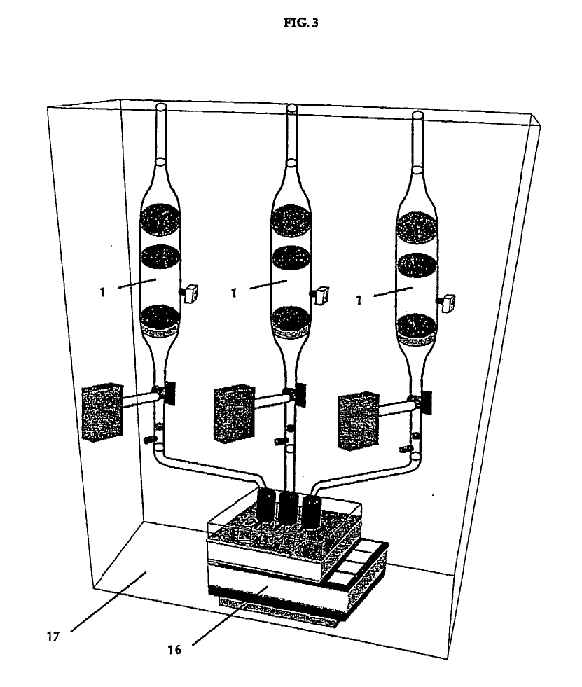

FIG. 3 an embodiment of the sample preparation device of the invention

FIG. 1 shows a first embodiment of the unit of the invention in the form of a cartridge. This cartridge is for preparing a reaction mixture without lysis and purification steps. The cartridge (1) has an inlet (2) and an outlet (3). A membrane (4) is present in the cartridge (1) and the composition, preferably the lyophilizate, is bound thereto. In addition, the cartridge (1) has an aperture (5) for applying a reduced pressure. For example, a tube from a vacuum pump can be connected to the aperture (5). A sample is put through the inlet (2) into the cartridge (1). The sample passes through the membrane (4) and thereby mingles with the composition. The finished reaction mixture then emerges through the outlet (3) and enters the collecting device (6).

FIG. 2 shows an embodiment of the unit of the invention in the form of a cartridge (1) which has additional devices for lysing and purifying a sample. A capillary (7) is optionally provided above the inlet (2) of the cartridge (1) in order to enable defined volumes to be introduced into the cartridge (1).

The sample first reaches a filter (8) having a pore size which does not allow intact cells to pass through. A lysis of the sample is then carried out. This can be achieved either by adding a lysing agent through the inlet (2) or by providing a filter (8) impregnated with lysing agent.

The lysed sample then passes through the filter (9) which is provided with a smaller pore diameter than filter (8). If volumes of more than 1 ml are processed, it is optionally possible to provide in the space between the filters (8) and (9) a solid-absorbing substance. The liquid thus proceeds onto the filter (10). The filter (10) is a filter which binds polynucleotides and which is configured as described above. The polynucleotides bind to the filter (10), while the remaining sample constituents penetrate through the filter (10) and reach the waste container (15) through the filter (11). The three-way tap (14) is set so that the substance passing through the cartridge (1) is guided into the waste container (15).

The three-way tap (14) is then set so that the substance passing through the cartridge (1) is now guided into the collecting device (6). A reduced pressure is applied to the cartridge (1) through the aperture (5) and destroys the membrane (12) of the supply unit. Eluent then reaches the cartridge (1) from the storage container (13). The eluent elutes the polynucleotides from the filter (10). The filter (10) may additionally be impregnated with substances which increase the surface tension, such as a polysilane, for example Dimeticon®. This impregnation results on the one hand in an additional filter effect. On the other hand, the impregnating agent is taken up by the eluate. This leads to the finished reaction mixture having a better penetration into liquid channels.

The eluate containing the polynucleotides passes through the filter (11) and reaches the membrane (4) to which the composition is bound. The eluate on passing through the membrane (4) takes up the composition, thus forming the finished reaction mixture. No reduced pressure should now be applied to the aperture (5), in order to avoid the reaction mixture passing through the aperture (5). The reaction mixture finally passes through the outlet (3) and reaches the collecting device (6).

FIG. 3 shows an embodiment of a sample preparation device of the invention. Three units such as, for example, cartridges (1) are disposed in an article (17), for example a polycarbonate block. These cartridges are connected to a reaction device (16) in such a way that the reaction mixture emerging from the respective cartridges (1) is supplied via lines to separate apertures of the reaction device (16). It is possible in this way for a plurality of reaction mixtures, in the present case three, to be prepared simultaneously and transferred into chambers of a reaction device (16). Subsequently, the cartridges (1) and the article (17) are separated from the reaction device (16). In the present case, the reaction device (16) is taken out and transferred into an apparatus for carrying out and evaluating a chemical reaction. As stated above, it is possible in another embodiment for the reaction device (16) to be disposed in the apparatus for carrying out and evaluating a chemical reaction even during the charging. In this case, the cartridges (1) and the article (17) are taken off the reaction device (16) after the reaction mixtures have been prepared.

It is possible with the present invention to prepare reaction mixtures for chemical reactions in a simple manner. In particular, the system is suitable as constituent of the procedure for chemical reactions which must pass through controlled temperature cycles. One example of such a reaction is the polymerase chain reaction (PCR) described at the outset.

Thus, the present invention facilitates the carrying out of methods intended to differentiate between different polynucleotides (DNA or RNA) present in a reaction mixture, or intended to detect various mutations in polynucleotides with the system of the invention. The system of the invention can thus be employed to detect protozoa, fungi, bacteria, viruses or particular DNA mutations. Besides the diagnosis of diseases or predispositions to diseases, the system of the invention can thus also be used in the area of pharmacogenomics, i.e. of therapy designed individually according to the genetic predisposition of the patient, or for phytochemistry, veterinary medicine, veterinary biochemistry, microbiology or generally for areas in which polynucleotide analysis is necessary.

### EXAMPLE 1

500 μl of whole blood were put dropwise into the cartridge of the invention. The blood was additionally mixed with EDTA in order to prevent the coagulation cascade and proteolysis. The whole blood was sucked through a prefilter (diameter 0.5 μm) in order to remove the coarsest particles. The sample was then passed through a nylon filter (diameter 0.5 μm) as second purification stage. The blood subsequently passed via a membrane (DEAE membrane/Hibond), which was impregnated on the underside with a Dimeticon solution, to the fourth filter, a wetting filter. After the blood had passed, the tap was turned from the “waste” setting to the “chip” setting. At the same time, the vacuum was increased until the membrane with the high-salt buffer (1M NaCl) burst. This buffer reached the wetting filter and then detached the nucleic acids from the membrane and led them through a second channel via a porous hard filter to whose underside the lyophilizate was bound. The lyophilizate had the composition described above for the reaction solution and had been obtained therefrom by conventional lyophilization methods. The amount of lyophilizate was chosen so that the final concentration resulting after wetting were 0.5 μmol/l primer, 0.2-0.4 μmol/l anchor and sensor probes, between 1-5 mmol/l magnesium chloride, appropriate for the problem for dNTP, dUTP-UDG (about 2 mmol/l), (5U) of 7 μl AmpliTaq gold polymerase, and 8% trehalose or mannose, 1.3% Carbopol 940, 1% Tween 20. After the solution had flowed through into the microchip in accordance with WO 03/019158 (or another analytical system), the solution was then ready for PCR analysis.

### EXAMPLE 2

Whole blood was put dropwise into the cartridge of the invention in analogy to example 1. The cartridge was then closed and heated at 95° C. for 5 minutes and centrifuged around its own axis for 10 minutes. A vacuum was then applied. It was possible to pass the plasma separated from the coagulum without high-salt solution directly through the wetting filter and the hard filter with the lyophilizate present thereon. After the solution had flowed into the microchip in accordance with WO 03/019158 (or another analytical system), the solution was then ready for PCR analysis.

### EXAMPLE 3

The whole blood sample was initially centrifuged at 2500 rpm for 10 minutes. 300 μl of the blood plasma obtained in this way were in one case mixed with EDTA and put dropwise into the cartridge of the invention, and in another case directly put dropwise into the cartridge of the invention. The sample passed through the filters described in example 1, coarse purification no longer being necessary. It was possible for the sample to pass without high-salt solution directly through the wetting filter and the hard filter with the lyophilizate present thereon. After the solution had flowed into the microchip in accordance with WO 03/019158 (or another analytical system), the solution was then ready for PCR analysis.

### EXAMPLE 4

In analogy to example 2, a urine sample instead of a whole blood sample was processed and delivered for PCR analysis.

### EXAMPLE 5

The samples obtained in example 1 to 4 were subjected to a conventional PCR reaction as described in WO 03/019158 and evaluated. Very reliable and accurate characterizations of polynucleotides were obtained.

1-25. (canceled)

26. A composition for carrying out chemical reactions, comprising
i) a solution comprising a polymerase;
ii) MgCl₂ and, where appropriate, at least one further alkali metal and/or alkaline earth metal halide;
iii) deoxyribonucleotide triphosphates (dNTPs);
iv) at least one primer;
v) a stabilizer;
vi) substances for detection of the reaction product; and
vii) where appropriate further additives.

27. The composition of claim 26, wherein said composition is a lyophilizate.

28. The composition of claim 26, wherein said chemical reaction is the polymerase chain reaction.

29. The composition of claim 26, wherein said polymerase is a DNA polymerase.

30. The composition of claim 29, wherein said DNA polymerase is Taq polymerase.

31. The composition of claim 26, wherein said further alkali metal metal halide is KCl.

32. The composition of claim 26, wherein said composition comprises two primers.

33. The composition of claim 26, wherein said stabilizer is a disaccharide.

34. The composition of claim 33, wherein said disaccharide is trehalose.

35. The composition of claim 26, wherein the substances for detection of the reaction product are fluorescent dyes.

36. The composition of claim 35, wherein said fluorescent dyes are bound to in each case an anchor oligonucleotide and a sensor oligonucleotide.

37. A unit for preparing reaction mixtures for chemical reactions, comprising an inlet and an outlet, and at least one support, wherein the composition of claim 26 is bound to at least one support.

38. The unit of claim 37, wherein said unit is a cartridge.

39. The unit of claim 37, wherein said chemical reaction is the polymerase chain reaction.

40. The unit of claim 37, wherein said support is a membrane.

41. The unit of claim 37, wherein said composition is a bound as lyophilizate to at least one support.

42. The unit of claim 37, wherein a device for applying elevated pressure or reduced pressure is provided.

43. The unit of claim 37, wherein a capillary is attached above the inlet.

44. The unit of claim 37, wherein one or more additional membranes or supports are present between the inlet and the support to which said composition is bound.

45. The unit of claim 44, wherein four additional membranes are present between the inlet and the support to which said composition is bound.

46. The unit of claim 37, wherein at least one additional support is designed so that polynucleotides can be bound thereto.

47. The unit of claim 46, wherein said additional support is a membrane.

48. The unit of claim 46, wherein said additional support is designed by providing diethylaminoethyl groups.

49. The unit of claim 47, wherein said additional membrane is impregnated with a substance which increases the surface tension of a liquid.

50. The unit of claim 49, wherein said substance which increases the surface tension of a liquid is a polydimethylsiloxane.

51. The unit of claim 44, wherein a substance which absorbs solids is provided in the space between two membranes.

52. The unit of claim 51, wherein said substance which absorbs solids is Aerosil.

53. The unit of claim 44, wherein the additional membrane located closest to the inlet is impregnated with a lysing agent.

54. The unit of claim 37, wherein a unit for supplying a liquid is provided above the support provided for binding polynucleotides.

55. The unit of claim 54, wherein said liquid is an eluent.

56. The unit of claim 37, wherein said unit for supplying a liquid is separated from the interior of the unit by a membrane, which can be made permeable on application of reduced pressure.

57. A method for preparing reaction mixtures for chemical reactions, comprising the steps:
a) introducing a sample into the unit of claim 37;
b) passing the samples through a support to which said composition is bound, so that the finished reaction mixture emerges from the outlet of the unit.

58. The method of claim 57, wherein said chemical reaction is the polymerase chain reaction.

59. The method of claim 57, wherein said support is a membrane.

60. The method of claim 57, wherein between steps a) and b) the additional steps of
a1) lysis of cells in the sample, and
a2) separation of the polynucleotides from other sample constituents are carried out.

61. The method of claim 60, wherein said step a1) takes place by contacting the sample with a membrane impregnated with lysing agent.

62. The method of claim 60, wherein said step a2) comprises binding the polynucleotides to a membrane, removal of the other sample constituents and subsequent elution of the polynucleotides from this membrane with the aid of an eluent.

63. The method of claim 62, wherein said eluent is a high-salt solution which comprises no chelating substances.

64. The method of claim 60, wherein in said step a2) the sample is guided through one or more membranes.

65. The method of claim 60, wherein in said step a2) the sample is guided through a substance which absorbs solids.

66. A device for preparing reaction mixtures for chemical reactions, comprising
a) at least one unit according to claim 62;
b) at least one reaction device which is connected via an aperture to the outlet of a unit and, after charging with a reaction mixture, can be separated from the sample preparation device.

67. The device of claim 66, wherein said chemical reaction is the polymerase chain reaction.

68. The device of claim 66, wherein said device comprises three units.

69. The device of claim 66, wherein said at least one unit is a cartridge.

70. The device of claim 66, wherein said at least one reaction device can, after the charging with a reaction mixture, be taken out of the sample preparation device and transferred into a device for carrying out and, where appropriate, evaluating said chemical reaction.

71. The device of claim 66, further comprising a device for carrying out and evaluating a chemical reaction, wherein said at least one unit can, after charging of said reaction device, be separated from the remaining device.

72. The device of claim 71, wherein said chemical reaction is the polymerase chain reaction.

73. A method for identifying polynucleotides in a sample, comprising the preparation of a reaction mixture for a polymerase chain reaction in a unit according to claim 62, transferring the reaction mixture into a reaction device and carrying out and evaluating a polymerase chain reaction.

74. The method of claim 73, wherein said polynucleotide is DNA.
